(19) United States
(12) Patent Application Publication
(10) Pub. No.: US 2009/0105127 A1
(43) Pub. Date: Apr. 23, 2009

(54) Methods for stabilizing corneal tissue

(75) Inventors: Vance Thompson, Sioux Falls, SD (US); Bruce DeWoolfson, Vienna, VA (US); Dale DeVore, Chelmsford, MA (US)

Correspondence Address: FINNEGAN, HENDERSON, FARABOW, GARRETT & DUNNER;LLP, 901 NEW YORK AVENUE, NW, WASHINGTON, DC, 20001-4413, US

(73) Assignee: Euclid Systems Corporation.

(21) Appl. No.: 12/285,683

(22) Filed: Oct. 10, 2008

Related U.S. Application Data
(63) Continuation-in-part of application No. PCT/US07/08049, filed on Apr. 3, 2007.
(60) Provisional application No. 60/791,413, filed on Apr. 13, 2006.

(51) Int. Cl. A61K 38/16

(52) U.S. Cl. 514/8

(57) ABSTRACT

Methods of stabilizing collagen fibrils in a cornea are disclosed. The stabilization may be effected by treating the cornea with a protein that crosslinks collagen fibrils, such as decorin. The stablization methods include treatment of corneas before, during, or after a surgical procedure, treatment of keratectasia, and treatment of keratoconus.

### RELATED APPLICATIONS

This application is a continuation-in-part of application No. PCT/US2007/008049, filed Apr. 3, 2007, which claims benefit of provisional application No. 60/791,413, filed Apr. 13, 2006, the contents of each of which is incorporated by reference in its entirety.

### FIELD OF THE INVENTION

The present invention relates to methods of stabilizing collagen fibrils in the cornea. These methods can be used to improve the outcome following refractive surgery, and to treat conditions of the cornea such as keratectasia and keratoconus.

### BACKGROUND OF THE INVENTION

The cornea is the first and most powerful refracting surface of the optical system of the eye. It is made up of five layers, the outermost of which is the epithelium. The epithelium is only four to five cells thick, and renews itself continuously. Underneath the epithelium is the acellular Bowman's membrane. It is composed of collagen fibrils and normally transparent. Below Bowman's membrane is the stroma. The stroma makes up approximately 90% of the cornea's thickness. This middle layer is mostly water (78%) and collagen (16%), although other proteoglycans and glycoproteins are also present. Descemet's membrane, which lies below the stroma, is also composed of collagen fibers, but of a different type than that found in the stroma. The endothelium lies beneath Descemet's membrane. It is a single layer of flattened, non-regenerating cells and functions to pump excess fluid out of the stroma.

When the cornea is misshapen or injured, vision impairment can result. In the case of a misshapen cornea, eyeglasses and contact lenses have traditionally been used to correct refractive errors, but refractive surgical techniques are now also routinely used. There are currently several different techniques in use. In radial keratotomy (RK), several deep incisions are made in a radial pattern around the cornea, so that the central portion of the cornea flattens. Although this can correct the patient's vision, it also weakens the cornea, which may continue to change shape following the surgery. Photorefractive keratectomy (PRK) is another technique. It uses an excimer laser to sculpt the surface of the cornea. In this procedure, the epithelial basement membrane is removed, and Bowman's membrane and the anterior stroma photoablated. However, regression and corneal haze can occur following PRK, and the greater the correction attempted, the greater the incidence and severity of the haze.

Laser in situ keratomileusis (LASIK) is yet another alternative. In this technique, an epithelial-stromal flap is cut with a microkeratome. The flap is flipped back on its hinge, and the underlying stroma ablated. The flap is then reseated. There is a risk that the flap created will later dislodge, however. In addition, the CRS-USA LASIK Study noted that overall, 5.8% of LASIK patients experienced complications at the three-month follow up period that did not occur during the procedure itself. These complications included corneal edema (0.6%), corneal scarring (0.1%), persistent epithelial defect (0.5%), significant glare (0.2%), persistent discomfort or pain (0.5%), interface epithelium (0.6%), cap thinning (0.1%) and interface debris (3.2%).

Most patients will have stable results after LASIK. That is, the one month to three month results will usually be permanent for most patients. However, some patients with initially good results may experience a change in their refraction over the first 3 to 6 months (and possibly longer). This shift in results over time is called regression. LASIK results in regression and haze less frequently than does PRK, presumably because it preserves the central corneal epithelium.

The chance of having regression following LASIK is related to the initial amount of refractive error: patients with higher degrees of myopia (−8.00 to −14.00) are more likely to experience regressions. For example, a −10.00 myope may initially be 20/20 after LASIK at the 2 week follow-up visit. However, over the course of the next 3 months, the refractive error may shift (regress) from −0.25 to −1.50 (or even more). This could reduce one's visual acuity without glasses to less than 20/40, a point at which the patient would consider having an enhancement.

All surgical procedures involve varying degrees of traumatic injury to the eye and a subsequent wound healing process. Netto et al., Cornea, Vol. 24, pp. 509-22 (2005). In addition, they reduce the eye's biomechanical rigidity, and postoperative keratectasia can result. Keratectasia is an abnormal bulging of the cornea. In keratectasia, the posterior stroma thins, possibly due to interruption of the crosslinks of collagen fibers and/or altered proteoglycans composition, reducing the stiffness of the cornea and permitting it to shift forward. Dupps, W. J., J. Refract. Surg., Vol. 21, pp. 186-90 (2005). The forward shift in the cornea causes a regression in the refractive correction obtained by the surgical procedure.

In the past several years there has been increasing concern regarding the occurrence of keratectasia following LASIK. In LASIK, the cornea is structurally weakened by the laser central stroma ablation and by creation of the flap. While the exact mechanism of this phenomenon is not completely known, keratectasia can have profound negative effects on the refractive properties of the cornea. In some cases, the cornea thins and the resultant irregular astigmatism cannot be corrected, potentially requiring PRK to restore vision. The incidence of keratectasia following LASIK is estimated to be 0.66% (660 per 100,000 eyes) in eyes having greater than −8 diopters of myopia preoperatively. Pallikaris et al., J. Cataract Refract. Surg., Vol. 27, pp. 1796-1802 (2001). Although at present keratectasia is a rare complication of refractive surgery, the number of procedures each year continues to increase, so that even a rare condition will impact many individuals. T. Seiler, J. Cataract Refract. Surg., Vol. 25, pp. 1307-08 (1999).

Keratoconus is another condition in which the rigidity of the cornea is decreased. Its frequency is estimated at 4-230 per 100,000. Clinically, one of the earliest signs of keratoconus is an increase in the corneal curvature, which presents as irregular astigmatism. The increase in curvature is thought to be due to stretching of the stromal layers. In advanced stages of keratoconus, a visible cone-shaped protrusion forms which is measurably thinner than surrounding areas of the cornea.

Keratoconus may involve a general weakening of the strength of the cornea, which eventually results in lesions in those areas of the cornea that are inherently less able to withstand the shear forces present within the cornea. Smolek et al., Invest. Ophthalmol. Vis. Sci. Vol. 38, pp. 1289-90 (1997). Andreassen et al., Exp. Eye Res., Vol. 31, pp. 435-41 (1980), compared the biomechanical properties of keratoconus and normal corneas and found a 50% decrease in the stress necessary for a defined strain in the keratoconus corneas. The alterations in the strength of the cornea in keratoconus appear to involve both the collagen fibrils and their surrounding proteoglycans. For example, Daxer et al., Invest. Ophthalmol. & Vis. Sci., Vol. 38, pp. 121-29 (1997), observed that in normal cornea, the collagen fibrils were oriented along horizontal and vertical directions that correspond to the insertion points of the four musculi recti oculi. In keratoconus corneas, however, that orientation of collagen fibrils was lost within the diseased areas. In addition, Fullwood et al., Biochem. Soc. Transactions, Vol. 18, pp. 961-62 (1990), found that there is an abnormal arrangement of proteoglycans in the keratoconus cornea, leading them to suggest that the stresses within the stroma may cause slipping between adjacent collagen fibrils. The slippage may be associated with loss of cohesive forces and mechanical failure in affected regions. This may be related to abnormal insertion into Bowman's structure or to abnormalities in interactions between collagen fibrils and a number of stabilizing molecules such as Type VI collagen or decorin Many of the clinical features of keratoconus can be explained by loss of biomechanical properties potentially resulting from interlamellar and interfibrillar slippage of collagen within the stroma and increased proteolytic degradation of collagen fibrils, or entire lamellae.

Because both keratoconus and postoperative keratectasia involve reduced corneal rigidity, relief from each disease could be provided by methods of increasing the rigidity of the cornea. For example, methods that increase the rigidity of the cornea can be used to treat postoperative keratectasia. Optionally, the treatment can be administered to a patient who plans to undergo a refractive surgical procedure as a prophylactic therapy. In other cases, the treatment can be administered during the surgical procedure itself. In still other situations, the treatment may not be initiated until after the refractive surgical procedure. Of course, various combinations of treatment before, during, and after the surgery are also possible.

It has also been suggested that a therapeutic increase in the stiffness of the cornea could delay or compensate for the softening of the cornea that occurs in keratoconus. Spoerl et al., Exp. Eye Res., Vo. 66, pp. 97-103 (1998). While acknowledging that the basis for the differences in elasticity between normal and keratoconus corneas is unknown, those authors suggest that a reduction in collagen crosslinks and a reduction in the molecular bonds between neighboring stromal proteoglycans could play a role.

As discussed below, the methods of increasing corneal rigidity and compensating for corneal softness that currently exist suffer from drawbacks that include development of corneal haze and scarring, and the risk of endothelial cell damage. These drawbacks are associated with the particular agents used in the methods. The need exists, therefore, for alternate methods of providing collagen crosslinks to increase the rigidity of the cornea.

The organization of collagen fibrils is the key to the cornea's transparency, and the arrangement of the collagen lamellae is the basis of its shape and strength. Meeks & Boote, Exp. Eye Res., Vol. 78, pp. 503-12 (2004), provide a recent review of the organization of the collagen fibrils and their associated proteoglycans. Each collagen fibril is made up of some 250 collagen molecules. Unlike collagen in other tissues, however, the axial periodicity is 65 nm rather than the usual 67 nm. The fibril diameter is approximately 31 nm, and each fibril is spaced on average about 62 nm apart, although this spacing varies, increasing from the central cornea towards the limbus. The fibrils are themselves organized into a lattice, but while early investigators predicted that the collagen fibrils would pack in a perfect lattice in the stroma, more recent studies have found that there are multiple lattices, each at most three fibril diameters.

Type I collagen is the predominant collagen within the fibrils, although types III, IV, V, VI, and XII are also present. Type VI collagen forms filaments that that run between the corneal fibrils and may interact with proteoglycans in the interfibrillar matrix to stabilize the fibrils. Proteoglycans also associate with the other collagen fibrils. There are two types of proteoglycans: chondroitin/dermatan-sulphate-containing and keratan sulphate containing. Decorin is the only molecule of the first type, whereas there are three keratan sulphate containing proteoglycans: lumican, keratocan, and mimecan. Recent studies suggest that the three-dimensional arrangement involves a backbone of collagen fibrils enwrapped by a ring-like structure of proteoglycans which interconnect next-nearest neighbor collagen fibrils to form a lamella. Muller et al., Exp. Eye Res., Vol. 78, pp. 493-501 (2004).

The collagen fibrils in the scar tissue that forms following refractive surgery are disordered, resulting in corneal cloudiness. Kaji et al., J. Cataract Refract. Surg., Vol. 24, pp., 1441-46 (1998). In most patients this scar tissue heals over time. As the scar heals, the collagen fibrils become regular in size and orientation, and the proteoglycans content returns to normal. In keratoconus, the collagen fibrils are also disordered. A recent study suggests that the lamellae unravel from their limbal anchors, much like a piece of cloth rips starting from a tear in the edge. Meek et al., Invest. Ophthalmol. & Vis. Sci, Vol. 46, pp.1948-56 (2005). Those authors also propose that part of this breakdown is triggered by a defect in the interfibrillar matrix that stabilizes the collagen fibrils, resulting in lamellar or fibrillar slippage.

Wollensak et al., J. Cataract Refract. Surg., Vol. 29, pp. 1780-85 (2003), have shown that the rigidity of the cornea can be improved by cross-linking the collagen fibers present in the cornea with the non-protein agent riboflavin. In their method, they apply a photosensitizing solution containing riboflavin to the cornea, then ultraviolet A (UVA) irradiation. This treatment forms collagen crosslinks that increase the rigidity of the cornea. In one preliminary study, the progression of keratectasia in patients with keratoconus was reduced. Wollensak et al., Am. J. Ophthalmol., Vol. 135, pp. 620-27 (2003), Although no adverse effects were observed in that clinical study, the investigators have found evidence of endothelial cell damage in a rabbit model following riboflavin/UVA treatment. Wollensak et al., J. Cataract Refract. Surg., Vol. 23, pp. 1786-90 (2003). In addition, the treatment also has the undesirable effect of inducing keratocyte apoptosis. Wollensak et al., Cornea, Vol. 23, pp. 43-49 (2004).

Aldehydes have also been used to crosslink collagen fibers in the cornea. For example, U.S. Pat. No. 6,537,545 describes the application of various aldehydes to a cornea in combination with a reshaping contact lens. The contact lens is used to induce the desired shape following either enzyme orthokeratology or refractive surgery, and the aldehyde is used to crosslink collagens and proteoglycans in the cornea. Spoerl & Seiler, J. Refract. Surg., Vol. 15, pp. 711-13 (1999), also tested the ability of several aldehydes to form collagen crosslinks. In application, however, aldehydes such as glutaraldehyde can lead to the development of corneal haze and scarring, while glyceraldehyde requires prolonged application times and its application is problematic. Wollensak et al., J. Cataract Refract. Surg., Vol. 29, pp. 1780-85 (2003).

Alternate methods of providing collagen crosslinks to increase the rigidity of the cornea are therefore needed.

It is accordingly an object of the invention to provide a method of stabilizing collagen fibrils. We have recently observed that small leucine-rich repeat proteoglycans (SLRPs), such as decorin; fibril-associated collagens with interrupted triple helices (FACITs); or the enzyme transglutaminase, can be used to retard relaxation of corneal tissue back to the original curvature when used as an adjunct to an orthokerotological procedure. See U.S. Pat. No. 6,946,440 to DeWoolfson and DeVore.

Although orthokeratology and surgical techniques such as LASIK each seek to improve visual acuity, they do so using radically different approaches. As a consequence, the mechanisms of corneal weakening are substantially different. Notably, the surgical techniques all involve at least some damage to the corneal structures and some tissue loss. Histological and ultrastructural investigations (Anderson et al. 2008) show minor epithelial in-growth into the flap wound, irregular collagen fibrils in the wound bed, and severed collagen bundles at the flap edge. Active wound healing processes were ongoing to repair damage induced during the LASIK procedure. Orthokeratology, in contrast, is a nonsurgical procedure to improve refractive errors of the eye involving the use of a series of progressive contact lenses that gradually reshape the cornea and produce a more spherical anterior curvature. The procedure is noninvasive; thus, unlike in LASIK, there is no associated damage to or thinning of the cornea. In orthokeratology, the cornea remains intact.

There are also fundamental differences between the cornea of an orthokeratology patient and the diseased cornea of a patient with keratoconus. As noted, keratoconus is a degenerative, and potentially blinding, corneal disease characterized by regions of stromal thinning spatially associated with cone-shaped corneal surface deformation. The cornea of the typical orthokeratology patient, in contrast, exhibits normal thickness and biomechanical strength.

Given these fundamental differences, it was not predictable that an agent employed during an orthokeratology procedure on an intact cornea of normal thickness could also be used before, during, or after a surgical procedure to improve the outcome of a surgical procedure that disrupted the cornea and removed corneal tissue, or that such an agent could be used to treat diseased corneas as occur in keratoconus.

Nevertheless, the inventors have now found that, despite the fact that surgery disrupts the cornea and removes corneal tissue, methods of stabilizing collagen fibrils using proteins that crosslink the collagen fibrils, such as decorin or the enzyme transglutaminase, may be used to improve the outcome following a surgical procedure to improve visual acuity. Those results also provide a basis for treating diseases of the cornea, such as keratectasia from other causes, and keratoconus.

### SUMMARY OF THE INVENTION

In accordance with the invention, methods of stabilizing collagen fibrils in a cornea are disclosed. These methods comprise administering to the eye of a patient a composition comprising a protein that crosslinks collagen fibrils and a pharmaceutically acceptable carrier. In one embodiment of the invention, a protein, such as decorin, crosslinks the collagen fibrils by binding to each of two different fibrils to form a bridge there between. In another embodiment of the invention, a protein, such as transglutaminase, crosslinks collagen fibrils by catalyzing the formation of a covalent bond between an amino acid in one collagen fibril and an amino acid in a second collagen fibri. In one embodiment of the invention, the collagen fibrils are stabilized in a cornea subject to a refractive surgical procedure. The stabilization treatment can be initiated either before, during, or after the surgery. The refractive surgical procedures include, but are not limited to, Radial Keratotomy (RK), Photorefractive Keratoplasty (PRK), LASIK (Laser-Assisted In Situ Keratomileusis), Epi-LASIK, IntraLASIK, Laser Thermal Keratoplasty (LTK), and Conductive Keratoplasty.

The invention also provides methods of treating keratectasia, comprising administering to the eye of a patient a composition comprising a protein that crosslinks collagen fibrils and a pharmaceutically acceptable carrier. The treatment can be prophylactic, contemporaneous with a surgical procedure, postoperative, or can involve multiple administrations during one or more of those time points. Although the keratectasia may develop following a refractive surgical procedure, it may also develop in an eye that has not had a surgical procedure. In one embodiment of the invention, the keratectasia develops following LASIK.

The invention also provides methods of treating keratoconus, comprising administering to the eye of a patient who has keratoconus a composition comprising a protein that crosslinks collagen fibrils and a pharmaceutically acceptable carrier.

In any of the methods of the invention, a protein that crosslinks collagen fibrils by binding to each of two different fibrils to form a bridge there between may be used. Decorin is one example of such a protein. Alternatively, or in addition, a protein that crosslinks collagen fibrils by catalyzing the formation of a covalent bond between an amino acid in one collagen fibril and an amino acid in a second collagen fibril can be used in any of the disclosed methods. Transglutaminase is an example of a protein that catalyzes formation of such covalent bonds.

Additional objects and advantages of the invention will be set forth in part in the description which follows, and in part will be obvious from the description, or may be learned by practice of the invention. The objects and advantages of the invention will be realized and attained by means of the elements and combinations particularly pointed out in the appended claims.

It is to be understood that both the foregoing general description and the following detailed description are exemplary and explanatory only and are not restrictive of the invention, as claimed.

### BRIEF DESCRIPTION OF THE DRAWINGS

FIG. 1 presents a histogram of post-LASIK corneal hysteresis in a patient that received decorin during the LASIK procedure in one eye (treated eye). The other eye was subjected to LASIK but did not receive the decorin treatment (untreated eye). The x-axis shows measurements taken as a baseline and at various time points post surgery. The Y-axis shows the results as a percentage of baseline.

FIG. 2 presents a histogram summarizing the results for a total of five myopic patients that received decorin during their LASIK procedure in one eye (treated eye). The other eye of each patient was subjected to LASIK but did not receive the decorin treatment (untreated eye). The x-axis shows measurements taken as a baseline and at various time points post surgery. The Y-axis shows the results as a percentage of baseline.

### DESCRIPTION OF THE EMBODIMENTS

The inventors have found that collagen fibrils in the cornea can be stabilized by administering to the eye one or more proteins that crosslinks the collagen fibrils even in a cornea subject to a surgical procedure in which the cornea is disrupted and tissue removed or in a diseased cornea. In order that the present invention may be more readily understood, certain terms are first defined. Other definitions are set forth throughout the description of the embodiments.

I. Definitions

A “refractive surgical procedure” includes, but is not limited to, Radial Keratotomy (RK), Photorefractive Keratoplasty (PRK), LASIK (Laser-Assisted In Situ Keratomileusis), Epi-LASIK, IntraLASIK, Laser Thermal Keratoplasty (LTK), and Conductive Keratoplasty.

“Stabilizing” includes increasing the rigidity, as measured by the Corneal Response Analyzer manufactured by Reichert Ophthalmic Institute. This instrument gives a quantitative measure of corneal rigidity called the Corneal Resistance Factor (CFR) and also a quantitative measure of corneal Historesis. “Stabilizing” can also mean decreasing the ability of one collagen fibril to move relative to another collagen fibril by virtue of increased intermolecular interactions.

“Crosslinks” includes the formation of both direct and indirect bonds between two or more collagen fibrils. Direct bonds include covalent bond formation between an amino acid in one collagen fibril and an amino acid in another fibril. For example, the transglutaminase family of enzymes catalyze the formation of a covalent bond between a free amine group (e.g., on a lysine) and the gamma-carboxamide group of glutamine. Transglutaminase thus is not itself part of the bond. Indirect bonds include those in which one or more proteins serve as an intermediary link between or among the collagen fibrils. For example, decorin is a horse-shoe shaped proteoglycan that binds to collagen fibrils in human cornea forming a bidentate ligand attached to two neighboring collagen molecules in the fibril or in adjacent fibrils, helping to stabilize fibrils and orient fibrillogenesis. Scott, J E, Biochemistry, Vol. 35, pages 8795 (1996).

A “protein that crosslinks collagen fibrils” includes proteins that form direct or indirect crosslinks between two or more collagen fibrils. Examples include decorin and transglutaminase. In certain embodiments, a protein that crosslinks collagen fibers is not a hydroxylase, such as lysyl oxidase or prolyl oxidase. Although not a protein, riboflavin is also excluded from the practice of the invention.

“Transglutaminase” includes any of the individual transferase enzymes having the enzyme commission (EC) number EC 2.3.2.13. Examples of human transglutaminase proteins include those identified by the following REFSEQ numbers: NP_{—}000350; NP_{—}004604; NP_{—}003236; NP_{—}003232; NP_{—}004236; NP_945345; and NP_{—}443187. Besides human transglutaminase, transglutaminase prepared from non-human sources is included within the practice of the invention. Examples of non-human sources include, but are not limited to, primates, cows, pigs, sheep, guinea pigs, mice, and rats. Thus, in one embodiment, the transglutaminase is a transglutaminase solution prepared from an animal source (e.g., Sigma Catalogue No. T-5398, guinea pig liver). In other embodiments, however, the transglutaminase is from a recombinant source, and can be, for example, a human transglutaminase (e.g., the transglutaminase available from Axxora, 6181 Cornerstone Court East, Suite 103, San Diego, Calif. 92121 or from Research Diagnostics, Inc., a Division of Fitzgerald Industries Intl, 34 Junction Square Drive, Concord Mass, 01742-3049 USA.)

“Decorin” includes any of the proteins known to the skilled artisan by that name, so long as the decorin functions as a bidentate ligand attached to two neighboring collagen molecules in a fibril or in adjacent fibrils. Thus, “decorin” includes the core decorin protein. In particular, decorin proteins include those proteins encoded by any of the various alternatively spliced transcripts of the human decorin gene described by REFSEQ number NM_{—}001920.3. In general, the human decorin protein is 359 amino acids in size, and its amino acid sequence is set forth in REFSEQ number NP_{—}001911. Various mutations and their effect on the interaction of decorin with collagen have been described, for example by Nareyeck et al., Eur. J. Biochem., Vo. 271, pages 3389-98 (2004), and those mutants that bind collagen are also within the scope of the term “decorin,” as is the decorin variant known as the 179 allelic variant, De Cosmo et al., Nephron, Vol. 92, pages 72-76 (2002). Decorin for use in the methods of the invention may be from various animal sources, and it may be produce recombinantly or by purification from tissue. Thus, not only human decorin, but decorin from other species, including, but not limited to, primates, cows, pigs, sheep, guinea pigs, mice, and rats, may also be used in the methods of the invention. An example of human decorin that can be used in the methods of the invention is the recombinant human decorin that is available commercially from Gala Biotech (now Catalant). Glycosylated or unglycosylated forms of decorin can be used.

As used herein, the terms “treatment,” “treating,” and the like, refer to obtaining a desired pharmacologic and/or physiologic effect. A treatment can administer a composition or product to a patient already known to have a condition. A treatment can also administer a composition or product to a patient as part of a prophylactic strategy to inhibit the development of a disease or condition known to be associated with a primary treatment. In the context of a surgical procedure, prophylactic treatment is any treatment administered to a patient scheduled to undergo a surgical procedure for the purpose of improving the outcome of that surgical procedure or otherwise reducing undesirable secondary effects associated with the surgical procedure. An example of a prophylactic treatment is the administration of an immunosuppressive agent to a patient prior to the transplantation of an organ or tissue. “Treatment,” as used herein, covers any treatment of a condition or disease in a mammal, particularly in a human, and includes: (a) inhibiting the condition or disease, such as, arresting its development; and (b) relieving, alleviating or ameliorating the condition or disease, such as, for example, causing regression of the condition or disease.

A “pharmaceutically acceptable carrier” refers to a non-toxic solid, semisolid or liquid filler, diluent, encapsulating material or formulation auxiliary of any conventional type. A “pharmaceutically acceptable carrier” is non-toxic to recipients at the dosages and concentrations employed and is compatible with other ingredients of the formulation. For example, the carrier for a formulation containing polypeptides preferably does not include oxidizing agents and other compounds that are known to be deleterious to polypeptides. Suitable carriers include, but are not limited to, water, buffer solutions such as Balanced Salt Solution, dextrose, glycerol, saline, cellulosics such as carboxymethylcellulose or hydroxypropylmethylcellulose, polysaccharides such as hyaluronic acid, and combinations thereof. The carrier may contain additional agents such as wetting or emulsifying agents, pH buffering agents, or adjuvants which enhance the effectiveness of the formulation. Topical carriers include liquid petroleum, isopropyl palmitate, polyethylene glycol, ethanol (95%), polyoxyethylene monolaurate (5%) in water, or sodium lauryl sulfate (5%) in water. Other materials such as anti-oxidants, humectants, viscosity stabilizers, and similar agents may be added as necessary. Other examples of pharmaceutically acceptable carriers are presented throughout the specification, including in the examples.

Pharmaceutically acceptable salts suitable for use herein include the acid addition salts (formed with the free amino groups of the polypeptide) and which are formed with inorganic acids such as, for example, hydrochloric or phosphoric acids, or such organic acids as acetic, mandelic, oxalic, and tartaric. Salts formed with the free carboxyl groups may also be derived from inorganic bases such as, for example, sodium, potassium, ammonium, calcium, or ferric hydroxides, and such organic bases as isopropylamine, trimethylamine, 2-ethylamino ethanol, and histidine.

The terms “individual,” “subject,” “host,” and “patient,” used interchangeably herein, refer to a mammal, including, but not limited to, murines, simians, humans, felines, canines, equines, bovines, porcines, ovines, caprines, mammalian farm animals, mammalian sport animals, and mammalian pets.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

II. Proteins that Crosslink Collagen Fibrils

Some proteins can crosslink collagen fibrils directly by forming covalent bonds between two or more collagen fibrils without the protein itself becoming part of the covalent bond. Transglutaminase, which includes any of the individual transferase enzymes having the enzyme commission (EC) number EC 2.3.2.13, is an example of a protein of this type. Transglutaminase catalyzes the formation of a covalent bond between a free amine group (e.g., on a lysine) and the gamma-carboxamide group of glutamine. Thus not itself part of the bond, transglutaminase instead forms a direct covalent link between two collagen fibrils.

In some methods of the invention, transglutaminase is prepared in 0.01M Tris buffer, pH 7.2. But any other pharmaceutically acceptable buffer may be used so long as it does not form a complex with calcium and prevent activation of transglutaminase. Buffer concentrations therefore generally range from between 5mM to 100 mM or from between 10 mM and 50 mM. In some embodiments of the invention, the buffer concentration is 10 mM. The buffer may also include CaCl₂ in concentrations that range from 5 mM to 50 mM, or from 20 mM to 35 mM. In certain embodiments of the invention, the buffer is a 25 mM CaCl₂ solution

Irrespective of the buffer solution chosen, when transglutaminase is the protein chosen, its concentration generally ranges from 1 to 100 units, but in some embodiments the concentration may be from 5 units to 50 units. In other embodiments of the invention, the concentration ranges from 10 units to 25 units per 50 mL of final enzyme solution. An example of one transglutaminase solution used in the methods of the invention is 0.01 M Tris buffer, pH 7.2, containing 25 mM CaCl₂ and from 10 units to 25 units of transglutaminase per 50 mL. Other proteins that catalyze formation of direct bonds between collagen fibrils may be used at these concentrations and in these buffers as well.

Collagen fibrils can also be crosslinked by indirect bonds. In these embodiments of the invention, one or more proteins serves as an intermediary link between or among the collagen fibrils. Decorin is an example of a protein that crosslinks collagen fibrils by indirect bonds.

For use in the methods of the invention, decorin is generally dissolved or suspended in a physiologically compatible buffer solution. The concentration of decorin may range from about 10 to about 1000 μg/ml. In some embodiments, the concentration ranges from about 50 to about 750 μg/ml, while in other embodiments it may be from about 100 to about 500 μg/ml. Other proteins that indirectly link collagen fibers by forming a bridge between or among collagen fibrils may be used at the concentrations described for decorin.

The buffer used as a carrier for a protein that forms an indirect crosslink between collagen fibrils is not critical and may be any of a number of pharmaceutically acceptable buffers, such as a neutral pH phosphate buffer. Other suitable buffers include HEPES, TRIZMA® (Sigma-Aldrich, but any other supplier of TRIS buffer should also be acceptable). The buffer will generally have a concentration from about 0.005 to 0.5M at a pH ranging from 6.5 to 8.5, although in some embodiments the pH is from about 6.8 to about 7.6.

An example of a decorin solution for use in the methods of the invention is one that is sterile and non-pyrogenic, and in which decorin is present at a concentration of 500 μg/ml and is buffered with 10 mM sodium phosphate plus 15 mM NaCl having a pH of 7.2.

III. Method of Administering Proteins that Crosslink Collagen Fibrils

Various methods can be used to apply a protein that crosslinks collagen fibrils to the corneal surface. In one embodiment, a solution comprising a protein that crosslinks collagen fibrils is applied to an applicator that is positioned on the corneal surface, generally following one or more pretreatment steps to dissociate epithelial cell junctures, as described in detail in provisional application No. 61/064,730, filed Mar. 24, 2008. A reservoir in the applicator allows the protein solution to penetrate a controlled area of the corneal surface. The reservoir also prevents the protein solution from flowing off of the corneal surface and onto surrounding ocular tissues. One applicator that can be used in the method is that described in provisional application No. 61/064,731, filed Mar. 24, 2008.

In other embodiments, the protein that crosslinks collagen fibrils may be applied directly to the stromal bed. This application method can be used, for example, in those embodiments involving surgery. In those embodiments, the formulation may be topically applied as an eyedrop directly onto the stroma while the surgical flap is laid back. Thus, in particular embodiments, drops of the solution containing the protein that crosslinks collagen fibrils may be applied to the stromal bed during a LASIK procedure. In addition, or as an alternative to stromal bed application, the drops may be applied to the back of the surgical flap while it is lifted.

When transglutaminase is used in the methods of the invention, it may be prepared using the following procedure. An inactive enzyme preparation is first prepared. For example, 10 units of transglutaminase can be added to 10 mL of Tris buffer, and mixed until the transglutaminase crystals dissolve. The resulting solution can then be diluted to 50 mL by adding sterile water and stored frozen until ready to use. Activate enzyme may then be prepared by the addition of CaCl₂. Usually this is done just before application to corneal tissue. For example, 1 part CaCl₂ solution can be added to 10 parts transglutaminase solution. One mL of transglutaminase solution is usually sufficient for each application.

The methods of strengthening the cornea in association with a surgical procedure may be initiated at any of a variety of time points after the patient has been informed that surgery is needed, or informed that surgery is an option for that patient. For example, a patient considering LASIK may receive the strengthening treatment at the time of his or her LASIK prescreening examination. Alternatively, the strengthening treatment may be administered at a time between the prescreening exam and the surgery. In general, the strengthening treatment will take place within the month preceding the surgery, but of course in some cases the time period may be more than a month before the surgery. For example, it is possible that the strengthening treatment could be administered 5, 6, 7, 8, or even more weeks before. Usually, however, the strengthening treatment will be administered about one to two weeks before the corneal surgery. Often, when it is administered before surgery, the strengthening treatment will be administered about 10 days before the surgery, although it may be administered about 9, about 8, about 7, about 6, about 5, about 4, about 3, about 2, or about 1 days before the corneal surgery. It is also possible to treat the cornea on the same day as the corneal surgery.

In other embodiments, the strengthening treatment takes place during the surgical procedure. These embodiments do not exclude treatments at other times, such as before and/or after the surgical procedure. As noted, treatment during the procedure may take the form of the application of drops of a formulation containing a protein that crosslinks collagen fibrils, such as decorin, directly to the stromal bed while the surgical “flap” is lifted, for example, as in a LASIK procedure. The flap is then reseated. In certain embodiments, one or more drops may optionally also be applied to the back of the surgical flap before it is reseated. In other embodiments, one or more drops are applied to the back of the surgical flap without application of drops to the stromal bed.

Varying numbers of drops containing a protein that crosslinks collagen may be used when the strengthening treatment takes place during the surgical procedure. The number of drops administered, whether to the stromal bed, the surgical flap, or to both the stromal bed and the surgical flap will depend at least in part upon the concentration of the crosslinking protein in each drop and the drop volume. In one embodiment, two drops are applied to the stromal bed and one drop is applied to the back of the surgical flap before it is reseated. Other combinations of drop number are certainly possible and may be left to the discretion of the practitioner during individual procedures.

When the strengthening procedure takes place before or after the surgical procedure, or when a surgical procedure is not involved, the protein that crosslinks collagen fibrils may be applied topically. In those embodiments that involve application at a time other than during the surgical procedure, it may be desirable to control the application so that it is directed to the corneal surface. In those embodiments, an applicator such as that described in provisional application No. 61/064,731, filed Mar. 24, 2008, may optionally be used. When application is limited to the corneal surface, it is also generally desirable to pre-treat the cornea with agents that dissociate epithelial cell junctures to enhance penetration, particularly if the applied protein is a relatively high molecular weight protein. Such methods are described in detail in provisional application No. 61/064,730, filed Mar. 24, 2008. Each of provisional application No. 61/064,730 and No. 61/064,731 is incorporated by reference in its entirety.

The methods have been described generally with respect to their method steps and the compositions used. Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

It must be noted that as used herein and in the appended claims, the singular forms “a”, “an” and “the” include plural referents unless the context clearly dictates otherwise. Thus, for example, reference to “a subject polypeptide” includes a plurality of such polypeptides and reference to “the agent” includes reference to one or more agents and equivalents thereof known to those skilled in the art, and so forth.

Unless defined otherwise, all technical and scientific terms used herein have the same meaning as commonly understood by one of ordinary skill in the art to which this invention belongs. All publications mentioned herein, including patents, patent applications, and publications are incorporated herein by reference in their entireties to disclose and describe the methods and/or materials in connection with which the publications are cited.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

The invention described below is given by way of example only and is not to be interpreted in any way as limiting the invention.

Reference will now be made in detail to the present embodiments of the invention.

### EXAMPLE 1

### Transglutaminase Stabilizes the Shape of the Cornea Following Mechanical Deformation

Transglutaminase was studied in a series of ex vivo laboratory experiments on enucleated porcine cornea to optimize the effects of stabilization. Enucleated porcine eyes were placed in ice until treated. Prior to treatment, each eye was placed in a bracket for stability and subjected to topographical evaluation using the Optikon 2000 system. Six topographs of each eye were taken and true composites generated. The corneal surface was dried using sterile gauze and then wetted with drops of 0.02M disodium phosphate. The wetted eyes were again dried and exposed to drops of 0.02M disodium phosphate. A glass slide was balanced on the surface of the cornea. Solutions of transglutaminase and calcium chloride (CaCl₂) were prepared in 50 mM TRIS buffer, pH 8.5. The pH of TRIS buffer was adjusted to 8.5 by adding 2.5N sodium hydroxide (NaOH). Transglutaminase was prepared at 1 mg/mL in 10 mL of TRIS buffer. CaCl₂ was prepared at a concentration of 25 mM in 50 mL of TRIS buffer. Prior to administration, 1 mL of CaCl₂ solution was mixed with 9 mL of transglutaminase solution because transglutaminase requires Ca⁺⁺ as a catalyst. The transglutaminase/CaCl₂ solution was added dropwise to the area around the glass slide. Approximately 1 mL of enzyme solution was applied in a period of 2 minutes. The slide was then removed and the eye washed with 0.004M phosphate buffer, at pH 7.4. The eye was then reexamined topographically and photos taken. Following the topographical evaluation, the eyes were placed in Optisol for storage pending additional evaluations. Three eyes were treated using this protocol.

There were some difficulties in treating the first two eyes due to the difficulty in applying the enzyme solution while balancing the glass slide. In the third attempt, drops of transglutaminase were applied to the cornea and the slide applied to the corneal surface and held in place using thumb pressure. Drops of enzyme solution were subsequently applied to corneal surfaces around the glass slide. No flattening effect was noted in the first two eyes. The topographical results from the first eye appeared to show corneal steepening as shown in Table 1 below. However, topographical maps clearly demonstrated a flattening of the central cornea in the third eye. Refractive power was reduced by approximately 1.5 diopters. All eyes appeared clear by visual examination. Eyes were placed in Optisol for storage.

TABLE 1
Corneal Power as Measured by Topographical Mapping

| Porcine Eye No. | Pretreatment (in Diopter) | | Post-treatment (in Diopter) | |
|---|---|---|---|---|
| 1 | 38.98 | 37.33 | 42.14 | 39.2 |
| 2 | 39.89 | 37.7 | 39.98 | 37.26 |
| 3 | 40.25 | 38.16 | 38.79 | 36.83 |

After treatment, corneal buttons were dissected, placed in Optisol and shipped to Rutgers University for stress-strain analysis. In the stress-strain analysis, corneal buttons were placed on a slightly convex surface and exposed to compressive forces. Stress-strain curves represent the force per unit area of cross-section required to compress the cornea a certain amount as expressed in percentage. Resultant curves indicate several distinct phases. The lower part (low modulus region) represents the resistance to squeeze out fluid between collagen fibrils. The middle part, wherein the stress-strain curve does not change, and the upper part (high modulus region) represent compression of collagen fibrils. A reduction in low modulus indicates that the cornea is softer. An increase indicates that the corneal buttons are stiffer and have been stabilized.

Transglutaminase treatment gave encouraging results. Topographical evaluation indicated that one porcine eye treated with transglutaminase following corneal flattening using a glass slide exhibited a refractive power reduction of about 1.5 diopters after removal of the glass slide. Two additional eyes were included in this treatment series. Glass slides were also applied to these porcine eyes. However, enzyme addition was applied with the eyes in the horizontal position and did not appear to flow under the glass slide into the cornea. In these eyes, there was no evidence of a reduction in refractive power by topographical evaluation. Since these eyes did not show flattening of the central cornea following the application of the glass slide, it was unlikely that the corneal flattening observed in the successful eye was solely a result of the application of the glass slide.

### EXAMPLE 2

### Toxicity Evaluation of Decorin in the Feline Eye

The purpose of the following evaluation was to determine if (1) there is toxicity associated with the use of decorin on the eye; (2) assess the penetration of decorin into the cornea; and (3) quantitate decorin in the cornea following exogenous application of decorin.

One, three, and five daily applications of decorin were assessed using female cats (6 months to 2 years of age) with normal corneas as the model system. The decorin was obtained as a dry powder (Sigma Chem. Co., Milwaukee, Wis.) and reconstituted in a 0.1 M phosphate buffer. In order to perform the microscopic evaluations, decorin was labeled with Oregon Green 514 using a commercially available kit from Molecular Probes.

Five cats were used in the study. Each cat was sedated prior to topical application of medication or photography of the eye. All animals received an ocular examination and photographs (whole eye, slit lamp, and endothelial cells) prior to treatment. Eyes were randomly assigned to a treatment group. The decorin was applied to the interior of a contact lens and the lens placed on the cat's eye. The lens remained on the eye for 10 minutes. All animals were observed briefly daily during the study. Three eyes were randomly assigned to a treatment or control group (1 eye). At least 2 more eyes were obtained for use as controls for each of the histograph, TEM, and confocal microscopy evaluations.

One eye from each of the three treatment groups was treated with Oregon Green 5149 labeled decorin.

Treatment group 1 eyes received one application of 50 μg of decorin in 100 μl buffer on day 1. Photographs and exams were obtained just after treatment and again on days 2, 4, and 8-post treatment. Exams and photos were then done weekly for the remainder of the month.

Treatment group 2 eyes received one application of 50 μg decorin in 100 μl buffer on days 1, 2, and 3. Photographs and exams were obtained just after treatment and again on days 2, 3, 5, and 8. Thereafter, exams and photos were obtained weekly for the remainder of the month.

Treatment group 3 eyes received one application of 50 μg decorin in 100 μl of buffer on days 1, 2, 3, 4, and 5. Photographs and exams were obtained daily and again on day 8. Thereafter, exams and photos were obtained weekly for the remainder of the month.

All animals were euthanized at one month. The eyes were enucleated. Each eye was cut in half. One half was fixed in formalin for histological analysis (H&E stain) of toxicity. The second half was further divided in half, one section was used for TEM visualization of the decorin, and the remainder was examined using confocal microscopy for those cats treated with labeled decorin.

The confocal micrograph results showed that decorin penetrates the corneal tissue of the eye. In addition, a cornea treated five times with decorin according to the treatment protocol above (treatment group 3) contained more collagen fibril-associated decorin than the untreated cornea. Initial qualitative analysis indicates that the decorin filaments in the treated eye appear longer and “fatter”. These “fatter” filaments were observed throughout the stromal sections, including the epithelial region, mid-stroma, and endothelial regions.

### EXAMPLE 3

### Measurement of Corneal Hysteresis in the Feline Model

The effects of decorin (human recombinant decorin provided by Catalent, Inc., Wisconsin) application on the biomechanical properties of the feline cornea were measured in five animals in a study performed at the Dartmouth-Hitchcock Surgical Research Center, Lebanon, N.H. Chemical agents were administered to the treated eyes to enhance decorin penetration and to dissociate proteoglycan bridges between collagen fibers, as referenced in paragraph [062]. The biomechanical integrity of the cornea was measured using the Reichert Ocular Response Analyzer (ORA). The ORA utilizes a dynamic by-directional applanation process to measure corneal hysteresis (CH).

Table 2 shows the results from this study.

TABLE 2
Stabilization of Cornea Biomechanical properties following
Application of Decorin Solution

| | Before | After decorin | After |
|---|---|---|---|
| Animal # | Decorin (CH) | Treatment (CH) | 21 Days |
| AHH3 | 5.50 | 7.43 | 7.50 |
| QJD4 | 3.90 | 6.30 | 6.90 |
| RAF6 | 3.13 | 5.18 | 6.20 |
| BEA4 | 3.65 | 4.80 | 6.20 |
| IRH6 | 7.98 | 8.03 | 5.80* |

*suspect data

As shown, application of decorin solution substantially increased the biomechanical integrity, i.e. stability of the cornea in the treated feline eyes.

### EXAMPLE 4

### Ocular Irritation Studies in Humans

In safety trials with live human subjects in Shanghai, People's Republic of China in August 2004, 2.9 mg/ml of decorin in buffered saline solution was administered by the applicator method. To avoid discomfort from placing the applicator on the eye, proparican hydrochloride (0.5%) was first administered as an anesthetic. It appears that the clinician and the patient are most comfortable with a holding period of the applicator on the eye limited to about twenty (20) seconds, so that if the full dose cannot be delivered in that interval, repeat applications following one another over several minutes would be indicated. Work to date has been with a single application of a limited concentration of decorin only, however patients show no adverse effects and express no discomfort whatsoever. This type of safety study has been conducted on adolescent Chinese Ortho-K patients on three occasions. None of the safety tests suggest any adverse indications.

### EXAMPLE 5

### Measurement of Corneal Hysteresis in LASIK Patients

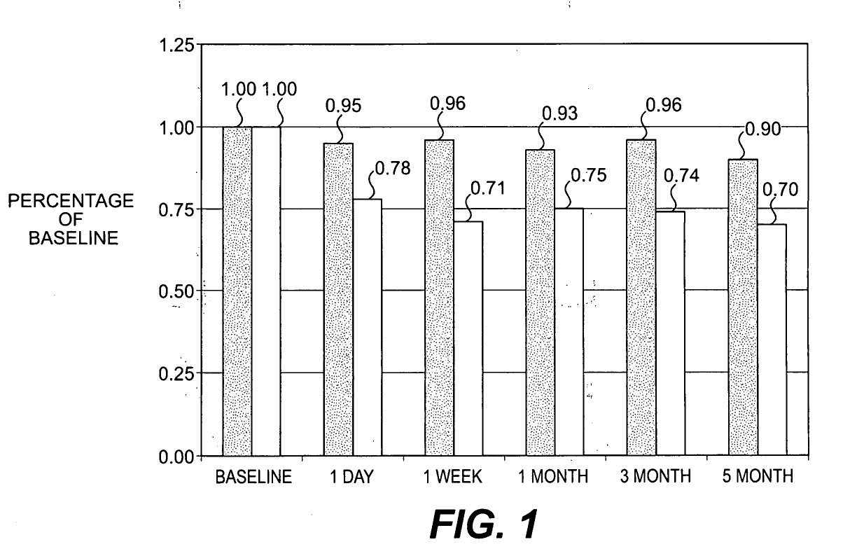

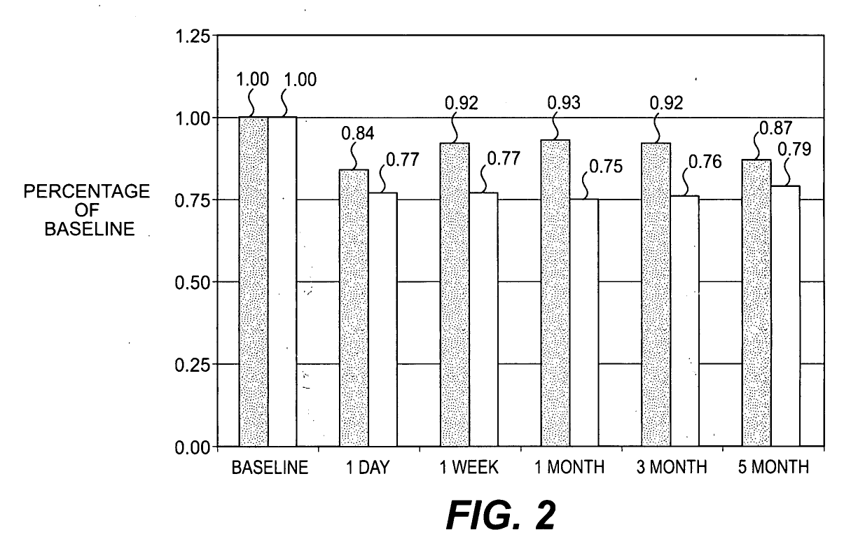

The effects of decorin application on the biomechanical properties of the post-LASIK cornea were measured in five human myopic LASIK patients in a pilot study performed by Gabriel Carpio, MD at the Hospital Angeles, Mexico. Two drops of decorin solution were applied to the stromal bed during the LASIK procedure and one drop to the back of the surgical flap. One eye was treated for each patient and the other eye served as control. The biomechanical integrity of the cornea was measured using the Reichert Ocular Response Analyzer (ORA). FIGS. 1 and 2 show the difference in corneal hysteresis (CH) between the treated eyes and the untreated eyes from the time of treatment through a five-month follow-up period. FIG. 1 presents the data for an individual patient, who had an OD of −6.25 and an OS of −6.00. That patient experienced an improvement of corneal hysteresis at each timepoint post-LASIK procedure when the treated eye was compared to the untreated eye (both relative to a baseline measurement). FIG. 2 groups the data for all five myopic patients. The grouped data also shows improvement of corneal hysteresis in the treated eyes relative to the untreated eyes (expressed relative to baseline) at all time points.

Based on these data, it should be possible to improve the outcome in refractive surgical procedures, such as LASIK. The preliminary results support improvements in corneal hysteresis of at least about 5%, 10%, 15%, or 20%, either when comparing the treated eye to an untreated eye of the same patient, or when comparing the same eye before and after treatment. It may also be possible to improve the hysteresis score of an eye subject to a refractive surgical procedure by at least 25%, at least 30%, at least 35%, or even more, compared to pre-treatment or a contralateral untreated eye. These results similarly suggest that it should be possible to obtain improvements in corneal hysteresis of at least about 5%, 10%, 15%, 20%, 25%, 30%, or 35%, or even more (relative to pre-treatment or a contralateral untreated eye) in corneas of patients with keratectasia or keratoconus. Furthermore, our data indicate that the improvement is present at the 1 day, 1 week, 1 month, 3 month, and 5 month time points. Thus, the various percentage improvement in hysteresis scores may be measurable at time points of at least 1 week, 1 month, 3 months, 5 months, or even more, such as 6 months, 9 months, 12 months, 18 months, 24 months, or 36 months.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A method of stabilizing collagen fibrils in a cornea of a refractive surgical patient, comprising administering a composition comprising a protein that crosslinks collagen fibrils and a pharmaceutically acceptable carrier to the eye of the patient during the refractive surgical procedure.

2. The method of claim 1, wherein the protein is decorin.

3. The method of claim 1, wherein the refractive surgical procedure is laser in-situ keratomileusis (LASIK).

4. The method of claim 3, wherein the protein is applied directly to the stromal bed while the surgical flap is lifted.

5. The method of claim 4, wherein the protein is decorin.

6. The method of claim 3, wherein the protein is applied to the back of the surgical flap while the flap is lifted.

7. The method of claim 6, wherein the protein is decorin.

8. The method of claim 3, wherein the protein is applied to the stromal bed and to the back of the surgical flap while the surgical flap is lifted.

9. The method of claim 8, wherein the protein is decorin.

10. A method of stabilizing collagen fibrils in a cornea of a refractive surgical patient, comprising administering a composition comprising a protein that crosslinks collagen fibrils and a pharmaceutically acceptable carrier to the eye of a patient who is scheduled to undergo a refractive surgical procedure.

11. The method of claim 10, wherein the protein is decorin.

12. The method of claim 10, wherein the refractive surgical procedure is laser in-situ keratomileusis (LASIK).

13. A method of stabilizing collagen fibrils in a cornea of a refractive surgical patient, comprising administering a composition comprising a protein that crosslinks collagen fibrils and a pharmaceutically acceptable carrier to the eye of a patient who has undergone a refractive surgical procedure.

14. The method of claim 13, wherein the protein is decorin.

15. The method of claim 13, wherein the refractive surgical procedure is laser in-situ keratomileusis (LASIK).

16. A method of treating keratectasia, comprising administering to the eye of a patient who has keratectasia a composition comprising a protein that crosslinks collagen fibrils and a pharmaceutically acceptable carrier.

17. The method of claim 16, wherein the protein is decorin.

18. The method of claim 16, wherein the keratectasia develops following a refractive surgical procedure that is laser in-situ keratomileusis (LASIK).

19. A method of treating keratoconus, comprising administering to the eye of a patient who has keratoconus a composition comprising a protein that crosslinks collagen fibrils and a pharmaceutically acceptable carrier.

20. The method of claim 19, wherein the protein is decorin.
